(19) United States
(12) Patent Application Publication
(10) Pub. No.: US 2008/0255671 A1
(43) Pub. Date: Oct. 16, 2008

(54) Joint Devices

(76) Inventor: Hans Rudolf Kriek, Bussum (NL)

Correspondence Address: DIEDERIKS & WHITELAW, PLC, 12471 DILLINGHAM SQUARE, #301, WOODBRIDGE, VA, 22192, US

(21) Appl. No.: 11/884,729

(22) PCT Filed: Feb. 21, 2006
(86) PCT No.: PCT/GB2006/000596

§ 371 Date: May 20, 2008

(30) Foreign Application Priority Data
Feb. 21, 2005 (GB) 0503536.5

(51) Int. Cl. A61F 2/38

(52) U.S. Cl. 623/20.29; 623/20.24; 623/20.14

(57) ABSTRACT

A joint device is disclosed for use as a prosthesis in a human or animal body. The joint device comprises a T-shaped member having a transverse pin fixedly connected to a column member and a pair of transverse sleeves configured to receive the pin and allow the pin to rotate about its longitudinal axis within the sleeves. The sleeves engage a bore in a first bone and the column member is coupled to a second bone so that the first and second bones are pivotably interconnected by the joint device. The sleeves are compressible so as to allow multi-directional movement of the bones in the joint.

The present invention relates to joint devices and to methods of replacing joint devices in human or animal bodies.

Replacement of worn out, damaged or diseased joints of human or animal bodies with artificial joint prostheses or components is an established medical procedure. Conventional joint prostheses or components for use in such procedures, which have been designed for virtually all types of joints, generally comprise component parts of the same or similar shape or form as all or part of the natural joint they are designed to replace. For example, the condyle surfaces of a knee joint may be replaced with metal plates of the same shape as the surfaces they replace. Alternatively, a whole joint may be replaced with a metal joint prosthesis taking a generally similar form to the original knee.

This conventional approach to joint replacements has various disadvantages. Firstly, the component parts of such joint prostheses are usually large and require invasive surgical procedures to implant the prosthetic components. Secondly, although the artificial component parts may have the same shape as the original natural joints, they cannot provide the same function as they are made from artificial material rather than bone and body tissues. For example, when the condyle surface of a knee is replaced the articular cartilage is removed and the new artificial surface will not be lubricated in the same way as a natural knee. This results in wear both of the artificial components and the body tissue surrounding and coming into contact with the artificial component.

WO 01/32109 discloses joint devices which replace the function of natural joints without using component parts having a similar shape or form as the joints whose function they replace.

The joint devices of WO 01/32109 comprise a pin and one or more pivoting member which rotates about the pin to enable the joint devices to pivotably interconnect two bones in a joint. In the joint devices comprising a single pivoting member the load on the joint is transmitted through only a single bearing surface in the joint device. Other joint devices are disclosed having two such bearing surfaces. However, these devices require multiple pivoting members to be implanted in the joint.

According to one aspect of the present invention there is provided a joint device for use as a prosthesis in a human or animal body, the joint device comprising a T-shaped member having a transverse pin fixedly connected to a column member, and a pair of transverse sleeves configured to receive the transverse pin and allow the pin to rotate about its longitudinal axis within the sleeves, wherein, in use, the pair of transverse sleeves engage a bore in a first bone, and the column member is coupled to a second bone so that the first and second bones are pivotably interconnected by the joint device.

The transverse pin and column member are preferably substantially fixed orthogonal to each other and the transverse sleeves receive the portions of the transverse pin either side of the region at which the pin is fixedly connected to the column member.

As the joint device of the present invention comprises a pair of transverse sleeves which rotatably receive the transverse pin of the T-shaped member the joint device pivotably interconnects two bones in the joint whilst providing two bearing surfaces between the sleeves and the transverse pin. This distributes the load on the joint device between multiple bearing surfaces and serves to reduce the wear on these surfaces. The joint device thus provides multiple bearing surfaces whilst requiring only a single column member connecting the two bones in the joint. This is advantageous over known joint devices which have two pivoting members connecting the bones in the joint as the joint device of the present invention is more simple and less invasive to implant than these known devices.

In the joint devices of the present invention the transverse sleeves and transverse pin co-operate so that the transverse pin can rotate about its own longitudinal axis within the sleeves. This rotation preferably provides the joint device with its major axis of rotation, rather than the rotation being provided by an interconnecting region of the transverse pin and a pivoting member.

In a preferred embodiment, the transverse pin and/or transverse sleeves are adapted to limit the rotation of the transverse pin about its own axis within the sleeves. Preferably, this limited rotation enables the column member, which is fixed to the transverse pin, to rotate within a range of 1000 in the plane perpendicular to the axis of the transverse pin.

The transverse sleeves preferably comprise at least one retaining means for fixedly securing them in the bore in the bone with which they are engaged. The retaining means may comprise a flange portion which is able to be screwed to the bone. Alternatively, or additionally, a rough or knurled surface, or an external screw threaded region may be provided on at least a portion of the surfaces of the transverse sleeves which engage the bore in the bone. Less preferably, the transverse sleeves may be cemented in place in the bone.

Preferably, at least the portions of the joint device which contact the body in which it is implanted are coated with, or formed of, a body compatible material. In a preferred embodiment, the components of the joint device which engage the bones may be coated with a material which stimulates bone growth.

The joint device of the present invention is preferably for use in joints which require a degree of movement/rotation about axes in addition to the major axis of rotation of the joint. For example, the joint device is preferably for implanting in a knee joint, in which the transverse sleeves and pin are implanted transversely across the femur and the column member is coupled to the tibia. Knee joints should ideally be able to bend a few degrees from side to side as viewed from the front (i.e. the anterior view). For example, in a natural knee joint the tibia may bend up to an angle of approximately ±7° relative to the axis of the femur. The joint device of the preferred embodiment accommodates for this ab-adduction pivotal movement, i.e. “side-to-side” movement. Natural joints may also have some degree of rotation around an axis which is substantially parallel to a longitudinal axis of a bone whose movement the joint facilitates. For example, a natural knee joint rotates around an endo-exo rotational axis passing through the medial condyle of the tibia and which is substantially parallel to the tibia. The joint device of the preferred embodiment may accommodate this “twisting” motion, which may be up to approximately ±5° in natural knee joints.

In order to provide for such multi-directional movement at least a portion of each transverse sleeve is preferably compressible to allow the transverse pin to rotate/move within the transverse sleeves to a limited extent in directions other than about its own longitudinal axis. Preferably, the transverse sleeves are configured so that the transverse pin can compress the sleeves such that the axis of the transverse pin is able to move/rotate relative to the axis through the sleeves. In a preferred embodiment this allows the column member, which is fixedly connected to the transverse pin, to rotate/move to a limited extent so that, for example, the tibia can perform limited side-to-side movement relative to the femur. Alternatively, or additionally, the transverse sleeves are configured so that the transverse pin can compress the sleeves such that the column member is able to rotate to a limited extent about its own longitudinal axis, for example, to provide for endo-exo rotation.

In a preferred embodiment each transverse sleeve is comprised of an outermost rigid layer or sub-sleeve and an inner compressible layer or sub-sleeve. Preferably, a further innermost layer or sub-sleeve is provided which is rigid and which receives a portion of the transverse pin. The innermost sleeve is sized and configured to allow the transverse pin to rotate about its own longitudinal axis within it. The innermost layer or sub-sleeve is preferably cylindrical whereas in the preferred embodiment the compressible and outermost layers or sub-sleeves are preferably conical.

Preferably, the joint device further comprises a column sleeve which receives a portion of the column member. The column member is preferably able to rotate to a limited extent about its own longitudinal axis within the column sleeve.

Therefore, according to another aspect the present invention provides a joint device for use as a prosthesis in a human or animal body, the joint device comprising a transverse pin and a column member for pivotably interconnecting two bones in a joint, wherein the joint device further comprises a column sleeve which is configured to engage a bore in one of the bones and to receive a portion of the column member so that the column member can rotate at least to a limited extent about its longitudinal axis within the column sleeve.

The column member is preferably able to rotate about its own axis within the column sleeve up to a predetermined angle.

The joint device of the preferred embodiment represents a mechanically convenient system which is able to provide substantially all of the directions of rotation/movement which would be provided for in a natural joint. The preferred joint device is advantageous in that it provides a generally more simple and more compact device than known devices which provide for multi-directional movement. Therefore, the joint device can be positioned with less removal of bone and tissue from the area of the joint, resulting in less trauma to the joint than when implanting conventional devices.

In the embodiments comprising a column sleeve the transverse pin and column member are preferably rigidly interconnected and the joint device comprises a pair of transverse sleeves as described above. In an alternative, less preferred embodiment, the transverse pin and column member are not rigidly interconnected and may be interconnected by means allowing rotation of the column member about the transverse pin in the plane perpendicular to the longitudinal axis of the transverse pin. In this less preferred embodiment the column member may comprise an aperture through which the transverse pin passes, the aperture being slightly larger than the diameter of the transverse pin.

The column sleeve of the preferred joint device enables the column member to rotate about its own axis by up to ±10-15°. More preferably, for example, when the joint device is implanted in a knee joint, the vertical sleeve enables the tibia to rotate approximately 10° inwards, i.e. the anterior of the tibia rotates towards the medial side, and approximately 5° outwards, i.e. towards the lateral side.

In the preferred embodiment the column sleeve and/or column member are configured so that the rotation of the column member within the column sleeve is resilient. In one embodiment, portions of the column member and sleeve are recessed to provide a cavity between them. A compressible material is provided in the cavity to provide the desired level of resilience in the rotation of the column member within the sleeve.

In another embodiment a portion of the column member comprises a protrusion and a portion of the column sleeve comprises a cavity/slot which receives the protrusion. The protrusion and cavity/slot are sized and configured to limit the rotation of the column member about its own axis within the sleeve. Preferably, the cavity in the sleeve is compressible and co-operates with the protrusion so that the rotation of the column member within the sleeve is resilient. In a less preferred embodiment, the protrusion may be compressible instead of the cavity, or both the protrusion and cavity may be compressible. In yet a further alternative embodiment, the protrusion may be provided on the inner surface of the column sleeve and the cavity may be formed in the column member.

In a less preferred embodiment the column sleeve is configured so that it has a compressible portion which allows the axis of the column member to pivot/rotate to a limited extent relative to the axis of the column sleeve. In this embodiment, the compressibility of the column sleeve enables the bone in which the column member is implanted to rotate slightly about the column member in the plane of the T-shaped member, i.e. in a side-to-side direction.

In a preferred embodiment, the column sleeve is configured to distribute the force on the joint device throughout the bone in which the column sleeve is implanted. Preferably, the column sleeve has an open end for receiving the column member and a closed end upon which the column member rests in use. At least a portion of the column sleeve preferably tapers outwardly in a direction towards the sleeve opening, i.e. the outer diameter of the sleeve increases towards the sleeve opening. Additionally, or alternatively, the sleeve may comprise a flange portion at least partially surrounding the sleeve opening which, in use, rests on top of the bone in which it is implanted.

In yet another embodiment, the joint device comprises a sleeve ring which co-operates with the column sleeve to distribute the load on the joint device across the bone in which the column sleeve is implanted. The sleeve ring comprises a flange portion which, in use, rests on the bone surrounding the bore in which the column sleeve is received. The sleeve ring further comprises an aperture for receiving the column sleeve. Preferably, the aperture in the sleeve ring is tapered and co-operates with a tapered portion on the column sleeve to support it. In this configuration the sleeve ring is able to at least partially transfer the load on the column member and its sleeve to the surface of the bone surrounding the bore.

In a preferred embodiment, one or more load distribution platforms may be provided which extend through the bone in which the column member is implanted so that the load transmitted through the column member is distributed across the bone. The load distribution platform preferably extends across the bone, i.e. substantially perpendicular to the length of the bone. The platform may extend substantially from the anterior to the posterior of the bone. Alternatively, the platform may extend substantially from the lateral to the medial side of the bone. The column member and/or its sleeve can engage the platform(s) when mounted in the bone such that load is distributed across the bone by the platforms.

In the preferred embodiment the column sleeve is secured in place in the bone by one or more screws passing transversely through the bone and a portion of the column sleeve. In this embodiment, the screws themselves may act as load distribution platforms.

In a further embodiment the joint device comprises an anti-subsidence structure for distributing the load on the bone in which the transverse pin is implanted. Preferably, the anti-subsidence structure comprises a tubular body portion which is configured to receive the transverse pin and a load distribution portion. Preferably, the tubular body portion comprises at least one opening through which an instrument can be inserted to force the load distribution portion away from the tubular body and towards the bone in which it is implanted.

As joints, even of the same type, have dimensions which vary the transverse pin and column member may be adjustable in length. For example, the column member may accommodate different distances between the transverse pin and the bone which the column member engages.

According to another aspect of the present invention there is provided a joint device for use as a prosthesis in a human or animal body, the joint device comprising a transverse pin and a column member, and a pair of transverse sleeves configured to receive the transverse pin, wherein in use the pair of transverse sleeves engage a bore in a first bone, and the column member is coupled to a second bone so that the first and second bones are pivotably interconnected by the joint device, and wherein the transverse sleeves are configured to be compressible to allow limited displacement of the rotation axis of the pin in use.

According to a yet further aspect, the present invention provides a method of replacing a human or animal joint comprising forming a bore in a first bone, inserting a transverse pin into the bore, fixing a pair of transverse sleeves in the bore around portions of the transverse pin, the transverse sleeves and pin being configured to allow the transverse pin to rotate about its own axis, coupling a column member with a second bone to be pivotably interconnected with the first bone, and fixedly interconnecting the transverse pin and column member.

In a preferred embodiment the column member is coupled to the second bone before the transverse sleeves are inserted into the bore around the transverse pin.

From another aspect the present invention provides a method of replacing a human or animal joint comprising forming a bore in a first bone, fixing a column sleeve in the bore, inserting a column member into the column sleeve, the column sleeve and column member being configured to allow at least limited rotation of the column member about its own axis, engaging a transverse pin with the column member and a second bone to be pivotably interconnected with the first bone.

As mentioned above, some previous knee devices or prostheses have required a relatively large portion of the knee joint to be removed. For example, the knee devices disclosed in WO 01/32109 comprise pins which bridge the intercondylar recess between the lateral and medial condyles of the femur. The pivoting member is then engaged with the pin within the intercondylar recess. In order for the pin to bridge the intercondylar recess the recess must be enlarged by reaming it out. Not only does this remove weight bearing regions of the joint between the bones but removal of such material also inhibits the natural functions of the joint such as lubrication.

In a preferred embodiment the bore which is formed in the femur for insertion of the transverse pin is formed above the intercondylar recess. A second bore is formed from the intercondylar recess at least part way into the femur so that it intersects with the first bore. The transverse pin and column member are able to be engaged with each other so that column member is pivotable in the region at which the first and second bores interconnect.

This method of replacing a knee joint is advantageous in that the transverse pin is inserted into a bore at least part of which is above the intercondylar recess rather than, for example, bridging such a recess. As the transverse pin does not bridge the recess it is not required to ream out a space large enough for both the transverse pin and column member. Therefore, the preferred method of knee joint replacement enables the knee to rotate about its approximate major axis of rotation whilst maintaining a high proportion of the natural joint for performing natural functions such as lubrication of the joint and bearing weight.

In a preferred embodiment, the transverse pin is mounted in the first bore formed in the femur such that a portion of the length of the transverse pin forms the upper surface of the intercondylar recess, i.e. the transverse pin is placed in the femur directly adjacent to the upper surface of the recess.

Various embodiments of the present invention will now be described, by way of example only, and with reference to the accompanying drawings, in which:

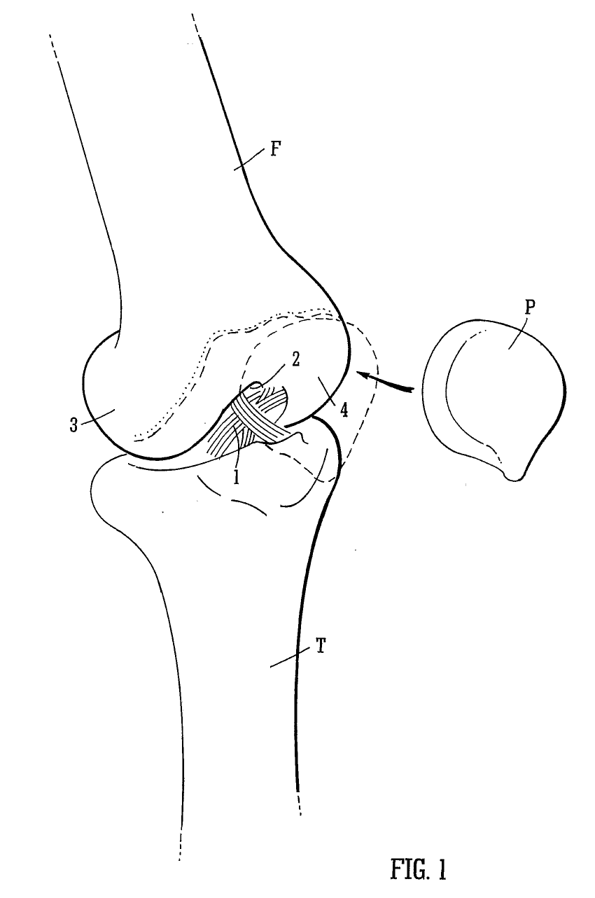

FIG. 1 shows a schematic of a natural knee joint;

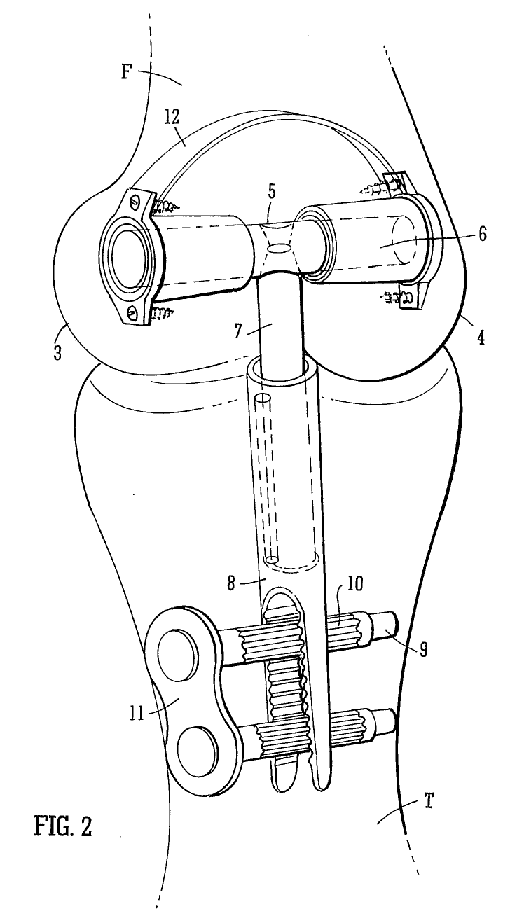

FIG. 2 shows a schematic of a knee joint including a joint device according to the preferred embodiment;

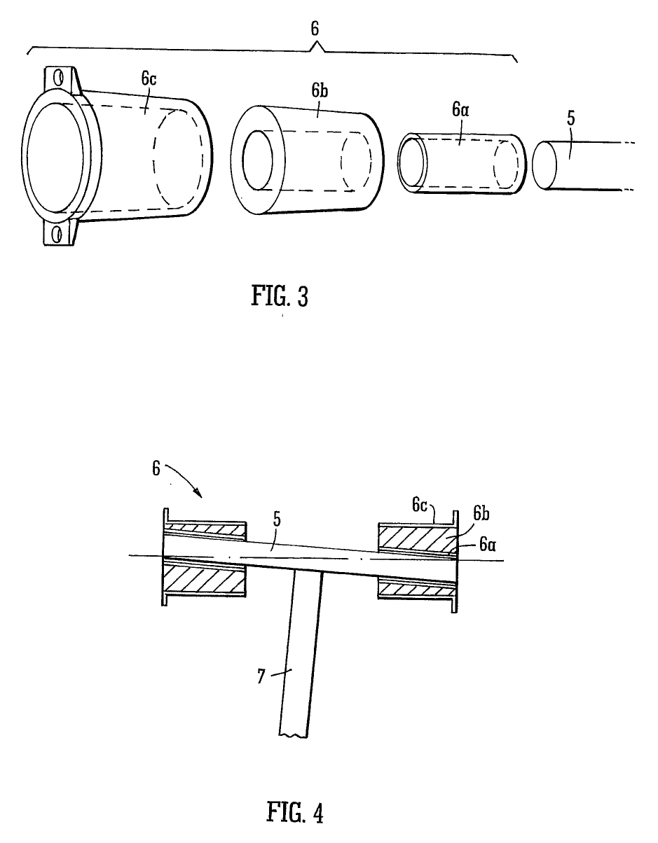

FIG. 3 shows a schematic of a portion of a transverse pin and the components of a transverse sleeve according to the preferred embodiment;

FIG. 4 illustrates the ability of the transverse pin to rotate within the transverse sleeves in an embodiment wherein the transverse sleeves comprise compressible members;

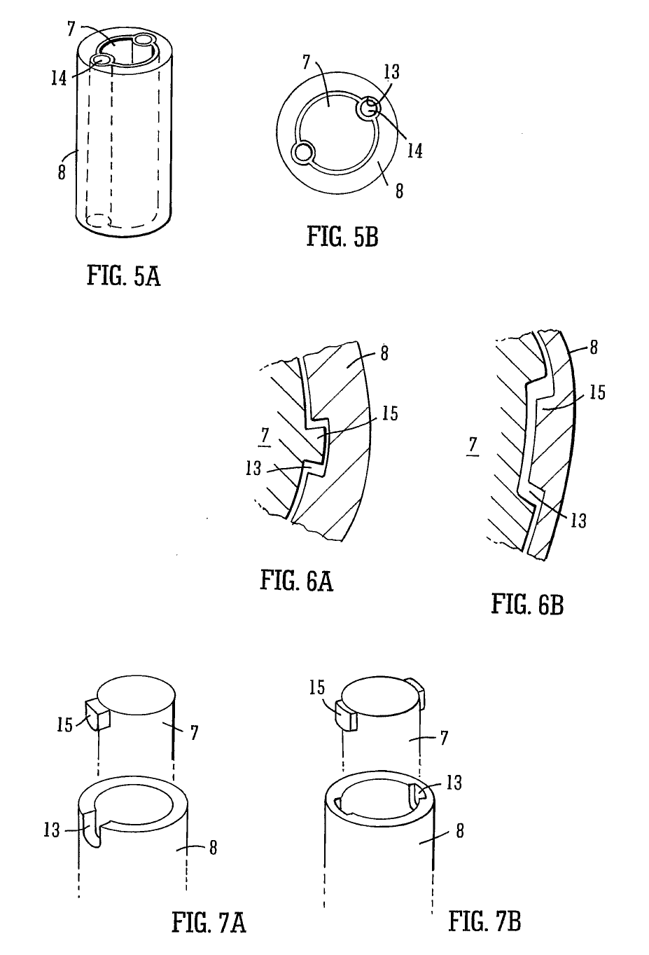

FIG. 5A shows a schematic of a portion of the column member and column sleeve according to a preferred embodiment and FIG. 5B shows a cross-section through the column member and sleeve;

FIGS. 6A and 6B illustrate portions of cross-sections through column members and sleeves according to embodiments wherein the column members and sleeves comprise protrusions or cavities;

FIGS. 7A and 7B show schematics of portions of the column members and sleeves wherein the sleeves do not comprise compressible elements;

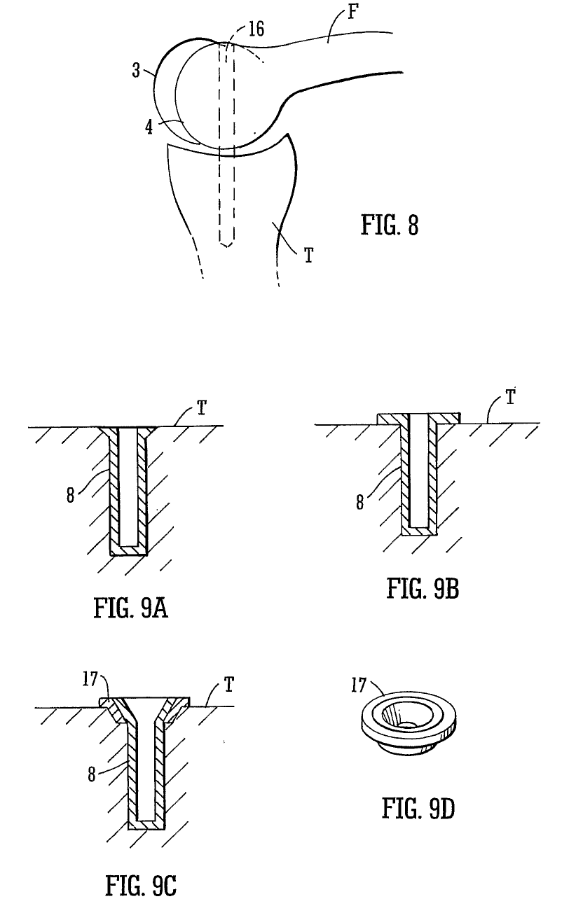

FIG. 8 shows a schematic of a knee joint which is bent to enable a bore to be formed in the tibia;

FIGS. 9A-9C show schematics of embodiments of column sleeves which are profiled to distribute the load of the joint over a larger area of the bone and FIG. 9D shows a schematic of an embodiment of a sleeve ring used in FIG. 9C; and

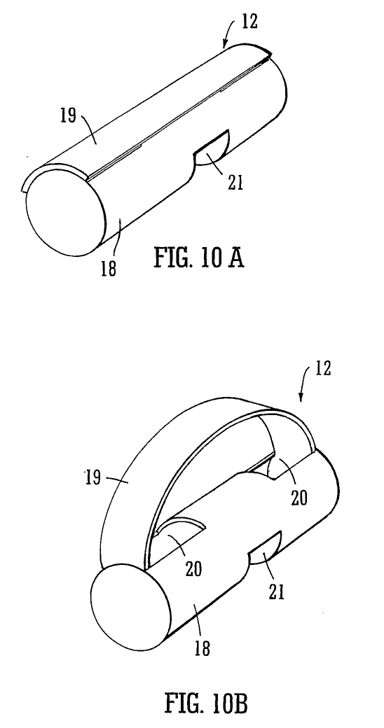

FIG. 10A shows an embodiment of an anti-subsidence structure configured to be inserted in the bone and FIG. 10B shows the anti-subsidence structure in its configuration when deployed in the bone.

The joint device of the present invention is preferably for implanting in knee joints. For the purpose of understanding this embodiment the knee joint will be briefly described with reference to FIG. 1. The knee joint is formed in the region of the leg between the femur F and the tibia T. Cruciate ligaments 1 extend between the femur F and the tibia T in a gap or recess 2 between the femoral condyles 3,4. The front, i.e. anterior, of the knee joint is covered by the patella P.

FIG. 2 illustrates a knee joint in which a preferred joint device has been implanted. The joint device comprises a transverse pin 5, which is implanted in the femur F, and a column member 7 which is implanted in the tibia T. The transverse pin 5 and column member 7 are interconnected in such a way that they cannot move or rotate relative to each other. The joint device comprises sleeves 6,8 which receive portions of the transverse pin 5 and column member 7. The outer surface of each sleeve 6,8 fixedly engages the bore in the bone in which it is implanted. Preferably, transverse sleeves 6 engage each end of the transverse pin 5 and holds the transverse pin 5 axially and radially in place in the femur F. A column sleeve 8 preferably engages a bore in the tibia T and receives an end portion of the column member 7. The transverse pin 5 and column member 7 and their respective sleeves 6,8 co-operate such that the transverse pin 5 and column member 7 can rotate about their own longitudinal axes within the sleeves 6,8. The joint device may also include an anti-subsidence structure 12 which is described in more detail below.

FIG. 3 shows the components of a preferred transverse sleeve 6 which may be used to hold one end of the transverse pin 5 in the femur F. In this particular embodiment the transverse sleeve 6 is comprised of three sub-sleeves 6a,6b,6c. An innermost sub-sleeve 6a is preferably provided which receives an end portion of the transverse pin 5. The innermost sub-sleeve 6a is preferably formed from polyurethane or a metal and is dimensioned and configured such that the transverse pin 5 can rotate about its own longitudinal axis within it.

The innermost sub-sleeve 6a is preferably received inside an intermediate sub-sleeve 6b that is made from a compressible material, for example, rubber or silicone. The intermediate sub-sleeve 6b is preferably received inside an outermost sub-sleeve 6c which is securely fixed to the femur F by any known technique. Preferably, the outermost sub-sleeve 6c is provided with one or more flanges comprising at least one opening which abuts against the femur F around the periphery of the bore in which the sleeve 6 is inserted such that the outermost sub-sleeve 6C can be screwed or otherwise secured to the femur F through the openings.

FIG. 4 illustrates how the compressible intermediate sub-sleeves 6b allow the transverse pin 5 to perform limited pivotal movement relative to the axis of the bore in the femur F (shown as a dashed line). In the example shown, the force exerted by the joint on the transverse pin 5 has caused the lower region of the compressible sub-sleeve 6b on the right side of the joint device to be compressed and the upper region of the compressible sub-sleeve 6b on the left side to be compressed. This enables the transverse pin 5 to pivot to a limited extent in a clockwise direction within the bore in the femur F. Similarly, the compressible sub-sleeves 6b also allow the transverse pin 5 to rotate in an anti-clockwise manner. As the column member 7 is coupled to the transverse pin 5 the preferred joint device allows the tibia T to have a limited amount of side-to-side movement relative to the femur F in the lateral-medial direction, i.e. the joint device provides ab-adduction pivotal movement. As such, the joint device is able to mimic the limited lateral-medial movement that a natural joint provides.

The material, compressibility, shape and thickness of the compressible sub-sleeves 6b may be chosen so that the joint device provides the desired amount of side-to-side movement. For example, the compressible sub-sleeves 6b are preferably conical having a substantially constant inner diameter and a thickness which increases in a direction towards the outer ends of the transverse pin 5. The outermost sub-sleeve 6c may also be conical to house such a conical compressible sub-sleeve 6b. It is contemplated herein that the compressible sub-sleeves 6b may also allow limited pivotal movement of the transverse pin 5 in directions other than in the plane of the T-shaped member. For example, preferably the compressibility of the sub-sleeves 6b enables the ends of the transverse pin 5 to rotate into and out of the paper as viewed in FIG. 4, i.e. the tibia T is able to twist relative to the femur F to a limited extent to perform endo-exo rotational movement. The compressibility of the different regions of the compressible sub-sleeves 6b may vary according to the desired level of movement to be provided for in each direction.

In a preferred embodiment the transverse pin 5 and column member 7 are substantially cylindrical and may be made from titanium or a cobalt/chromium alloy. The innermost sub-sleeve 6a has an inner diameter sized so that the transverse pin 5 can rotate about its own axis within it. The innermost sub-sleeve 6a is preferably substantially cylindrical. The intermediate compressible sub-sleeve 6b preferably has a constant internal diameter which is sized to receive the innermost sub-sleeve 6a, preferably so that the innermost sub-sleeve 6a cannot rotate. The outer diameter of the intermediate compressible sub-sleeves 6b preferably increases in an axial direction towards the ends of the transverse pin 5. The outermost sub-sleeve 6c is also preferably conical and receives the intermediate compressible sub-sleeve 6b so that it does not rotate within it. The outermost sub-sleeve 6c is preferably made from titanium or a cobalt/chromium alloy.

It should be noted that although the various sub-sleeves 6a,6b,6c have been described as discrete components they may in fact be layers of a composite transverse sleeve 6. Further, any number of sub-sleeves or layers may be used in any order to perform the functions described above. In certain embodiments, the sleeves 6 for the transverse pin 5 are not required to comprise compressible components and in such embodiments the sleeve 6 may be formed from a single sleeve 6c.

FIG. 5A shows a preferred embodiment of a portion of the column member 7 arranged inside a column sleeve 8 and FIG. 5B shows a cross-section through the column member 7 and sleeve 8. As can be seen from FIGS. 5A and 5B, the column member 7 is preferably substantially cylindrical except for grooves which extend along at least part of its length 7. The column sleeve 8 preferably has a substantial constant inner diameter which is slightly larger than the outer diameter of the column member 7 to facilitate rotation of the column member 7 about its own axis within the sleeve 8. The inner surface of the sleeve 8 preferably has two grooves extending along at least a portion of its length, wherein the position and length of the grooves correspond to those on the column member 7 so as to cooperate to form cavities 13. Compressible strips of material 14 are arranged in the cavities 13 defined by the grooves. The column sleeve 8 is rigidly fixed in a bore in the tibia T. The compressible strips 14 are arranged such that the column member 7 can compress the strips 14 and rotate about its own longitudinal axis up to a predetermined extent within the sleeve 8. The compressible strips 14 are preferably cylindrical and may be formed from silicone or rubber. It is contemplated herein that any shape, size, length and number of cavities 13 and compressible strips 14 may be selected to provided the desired level of resilience in the rotation of the column member 7 within the sleeve 8.

FIGS. 6A and 6B show portions of cross-sections through other preferred column members 7 and sleeves 8. Referring to FIG. 6A, the column member 7 may comprise a protrusion 15 extending from its outer surface. The protrusion 15 may extend part or the whole of the length of the column member 7 that is within the column sleeve 8. The sleeve 8 has a cavity 13 for receiving the protrusion 15 on the column member 7. Preferably, a layer of compressible material is provided on the sleeve 8 within the cavity 13. Alternatively, the sleeve 8 may be provided with an inner compressible layer which has a cut out region forming the cavity 13. The cavity 13 is preferably configured so that when the column member 7 rotates about its own longitudinal axis the compressible layer provides a gradual increase in resistance to the rotation. In a less preferred embodiment the cavity 13 in the sleeve 8 may be relatively rigid and the protrusion 15 on the column member 7 may be formed from a compressible material.

FIG. 6B shows an embodiment of the column member 7 and sleeve 8 similar to that of FIG. 6A except that the protrusion 15 is provided on the column sleeve 8 and the cavity 13 is provided in the column member 7.

FIGS. 7A and 7B show portions of column members 7 and sleeves 8 according to less preferred embodiments in which compressible members are not provided. The column members 7 are provided with one or more protrusions 15 which are received in one or more slots/cavities 13 in the vertical column 8. The relative circumferential sizes of the protrusions 15 and slots/cavities 13 are selected so that the column members 7 can rotate about their own axes within the sleeves 8 up to a predetermined angle.

In other less preferred embodiments one or more compressible member may be provided along a length of the column member 7 and/or sleeve 8 such that the axis of the column member 7 is able to move relative to the axis of the bore in the tibia T in a similar manner to that described with respect to the transverse pin 5 in the transverse sleeves 6 in the femur F. In these embodiments the column member 7 and/or sleeve 8 is able to provide for ab-adduction, i.e. limited sideways movement of the tibia T relative to the femur F in the lateral-medial direction.

The co-operating transverse pin 5, column member 7 and their sleeves 6,8 enable the preferred joint device to accommodate for limited movement or rotation of the bones in the joint about axes in addition to that of the major axis of rotation. For example, as has been described above, the preferred joint device is for implanting in a knee joint and enables limited endo-exo rotational movement and/or lateral-medial movement of the tibia T relative to the femur F, obviously in addition to rotation about the major axis of the knee. The degree to which the preferred joint device can accommodate these non-major rotations/movements may be selected by tailoring the compressibility and/or configuration of the sleeves.

Referring back to FIG. 2, in order to insert the joint device a first bore is formed transversely in the femur F, preferably above the gap or intercondylar recess 2 between the femoral condyles 3,4 (see FIG. 1). This bore is for housing the transverse pin 5 and its respective sleeves 6. In a particularly preferred embodiment, the first bore is formed such that a portion of the length of the first bore is at the interface between the femur F and the gap 2 between the femoral condyles 3,4. The first bore is preferably formed at a relatively small angle to the direction normal to the axis of the femur F, following the theoretical axis of flexion/extension, i.e. the axis from the medial epicondylus femoris to the lateral epicondylus femoris.

A second bore may then be formed substantially axially in the end of the femur F which is proximate the tibia T such that the second bore interconnects with the first bore. Most preferably, the second bore is formed by a minimally invasive procedure which involves bending the leg such that the surgeon can gain access to the lower end of the femur F. The second bore may then be formed axially into the femur F from a position on its lower end such that the second bore interconnects with the first bore. Advantageously, this method of forming the second bore enables the surgeon to avoid the ligaments 1 (see FIG. 1) between the tibia T and the femur F relatively easily. The second bore is preferably formed proximate and medial to the quadriceps tendon. The second bore provides the space necessary for the column member 7 rotate so that the knee joint can flex and extend.

A third bore may then be formed in the tibia T. The third bore is for housing the column sleeve 8 which receives the column member 7, or in a less preferred embodiment the third bore receives the column member 7 without any sleeve 8. Referring to FIG. 8, the third bore is preferably formed in the tibia T whilst the knee is bent. This may be performed by first forming a bore 16 through the medial condyle of the femur F, preferably at a point close to the patella groove. The bore 16 is preferably an extension of the second bore. An instrument used for boring may then be conveyed through the bore 16 to reach the upper surface of the tibia T. The third bore may then be formed in the tibia T. In this embodiment, the third bore in the tibia T is preferably smaller than the bore 16 through the medial condyle of the femur F and receives a column sleeve 8 and column member 7 sized and configured to be passed through the bore 16 in the medial condyle of the femur F.

It is contemplated herein that the first, second and third bores may be formed in any manner and in any order, i.e. the first or second or third bores may be formed in the order of first, second or third.

After the bores have been formed the column sleeve 8 may be inserted into the third bore in the tibia T. The column sleeve 8 may be fixed into the tibia T by any known means, for example, by providing a screw threaded region or a rough or knurled region on the outer surface of the sleeve 8. In the preferred embodiment the column sleeve 8 is fixedly secured in the tibia T by one or more screws 9 as shown in FIG. 2. In this embodiment the sleeve 8 comprises a lower portion for receiving the screws 9. Screw plugs 10 may be employed between the screws 9 and the sleeve 8 so that when the screws 9 are screwed into the screw plugs the plugs 10 expand and fix to the sleeve 8. A screw plate 11 is preferably provided against the outer side of the tibia T to prevent the screw heads from damaging the tibia T. The screws 9 also preferably assist in dispersing the load on the joint within the tibia T. The screws 9 may be introduced into the tibia T from any direction, although they are preferably introduced into the tibia T from the anterior or medially, or anterior-medially

In the preferred embodiment the articulation surfaces of the joint, for example the surfaces between the tibia T and femur F in the knee joint, do not contact each other once the knee device is implanted. As such, these surfaces may be prevented from being loaded. This is especially advantageous insofar as one or both of these surfaces demonstrate signs of degeneration, inflammation, arthritis or any other condition which might render them unsuitable for contacting each other.

In the preferred embodiment the cartilage/material on the surface of the tibia T and/or femur F between the two bones is reamed back to a certain extent so that the bones do not contact each other when they move relative to each other. The removal of cartilage/material may be achieved by access to the region between the bones via an incision in the anterior, medial or lateral side of the knee. Access to the region between the bones might also be gained via the bore 16 in the femur F as shown in FIG. 8.

FIGS. 9A-9C show longitudinal cross-sections through various embodiments of column sleeves 8. FIG. 9A shows an embodiment wherein the outer surface of the sleeve 8 tapers outwardly at the end into which the column member 7 is inserted. The bore in the tibia T preferably also tapers outwardly in the region near the opening to accommodate the tapering portion of the sleeve 8. The tapered configuration of the sleeve 8 assists in dispersing the load on the joint over a larger cross-sectional area of the tibia T.

FIG. 9B shows an embodiment of a column sleeve 8 having a flange portion which sits on top of the end surface of the tibia T in use. Preferably, the flange is circular and extends all of the way around the sleeve and bore openings.

FIG. 9C shows a further embodiment wherein a separate sleeve ring 17 is provided which co-operates with the column sleeve 8 to disperse the load on the joint within the tibia T. The sleeve ring 17 is shown in FIG. 9D and is preferably conical having tapered inner and outer surfaces and a flange which sits on the end of the tibia T in use. Preferably, the outer surface of the sleeve 8 is also tapered to co-operate with the taper on the inner surface of the sleeve ring 17. Such a sleeve ring is preferably inserted onto the opening of the bore in the tibia T from a position medial from the anterior side of the tibia T before the sleeve 8 is inserted through the sleeve ring 17 and into the bore.

After the sleeve 8 has been inserted into the tibia T the column member 7 is preferably introduced into it. The column member 7 may be passed through the bore 16 in the femur F and into the bore in the tibia T. In a preferred embodiment the transverse pin 5 is then passed into the transverse bore in the femur F. The transverse sleeves 6 are then preferably inserted into the ends of the transverse bore around the transverse pin 5. The outermost sub-sleeves 6c of the transverse sleeves 6 are secured in the bore so that they cannot move, preferably by screwing the flanges of the outermost sub-sleeves 6c into the lateral and medial sides of the femur F.

The transverse pin 5 comprises a region which interconnects with the column member 7. As can be seen from FIG. 2, in one embodiment this region comprises a channel through the transverse pin 5, wherein the diameter of the channel tapers outwardly from the centre of the transverse pin 5 to the outer surfaces of the pin 5. The upper portion of the column member 7 is preferably also tapered to be received in a tapered portion of the channel in the transverse pin 5. It is important that the column member 7 is unable to rotate about its own axis within the channel in the transverse pin 5. In one embodiment the column member 7 is fixedly connected to the transverse pin 5 by a screw. In this embodiment the column member 7 has an axial bore in the end portion that engages the transverse pin 5. The axial bore is preferably threaded so that it can receive a screw which is inserted into the channel in the transverse pin 5 from the opposite side of the transverse pin 5 to that which the column member 7 is inserted. The screw cooperates with the conical portion of the channel in the transverse pin 5 to pull the column member 7 into tight engagement with the transverse pin 5. In the preferred method the screw for fixing the transverse pin 5 and column member 7 together is conveyed through the bore 16 which is formed in the medial condyle of the femur F close to the patella groove (see FIG. 8).

In another embodiment the transverse pin 5 comprises two separate portions which interconnect with channels extending into opposite sides of the column member 7. The two portions of the transverse pin 5 are interconnectable with the column member 7 so that they cannot move relative to it and so that the portions of the transverse pin 5 essentially form a continuous pin. The two portions of the transverse pin 5 may be inserted into the transverse bore in the femur F from different sides, i.e. from the lateral and medial sides.

FIGS. 10A and 10B show an embodiment of an anti-subsidence device 12 which may be implanted into the transverse bore in the femur F before the transverse pin 5 and sleeves 6 are inserted into the bore. FIG. 10A shows the anti-subsidence structure 12 in the configuration that it is inserted into the transverse bore. The structure preferably comprises a substantially tubular body 18 and a load distribution platform 19. As can be seen from FIG. 10B the tubular body 18 of the structure comprises at least one opening 20 which enables an instrument to be inserted into the tubular body 18 to force the load distribution platform 19 away from the tubular body 18. In this manner the load distribution platform 19, which is preferably a metal structure, can be forced upwards into the cavity in the femur F above the transverse pin 5, as can be seen in FIG. 2. The tubular body 18 further comprises an opening 21 which allows the column member 7 to rotate about the major axis of rotation of the knee. In one embodiment the anti-subsidence structure 12 is configured so that the load distribution platform 19 is automatically forced upwards into the cavity in the femur F as the transverse pin 5 and/or sleeves 6 are inserted into the bore in the femur F.

The anti-subsidence structure 12 is configured so that it can distribute the load on the femur F over a larger area. This is particularly advantageous as the procedure to insert the joint device may lead to some of the bone in the cavity above the joint device being destroyed and re-absorbed by the body.

The transverse pin 5 and column member 7 are preferably formed of titanium, a cobalt/chromium alloy, chrome or polyetheretherketone (PEEK). In embodiments wherein one of the transverse pin 5 or column member 7 is not housed in a sleeve 6,8, self-tapping threads may be provided on portions of the transverse pin 5 or column member 7 for directly engaging the bore in the bone. The transverse pin 5 or column member 7 may have a minimum diameter of, for example, 5 to 10 millimetres which allows them to be sufficiently strong to support joints such as knee joints but not so large as to be too invasive on insertion into the bores in the bones.

Preferably, the transverse pin 5 has a length which allows it to extend between the medial and lateral side of the femur F above the gap 2 between the femoral condyles 3,4.

The column member 7 preferably fits between the anterior and posterior cruciate ligaments 1 and may be inserted without causing significant trauma or interfering with the function of these ligaments 1. This has the advantage of generally reducing trauma to the knee joint and improving recovery time.

The column member 7 and/or column sleeve 8 may extend through the tibia T to a load distribution platform or other load supporting device, such as load distribution screws 9. In this embodiment the bore in the tibia T extends at least to the load distribution platform. The column member 7 or its sleeve 8 may rest on a load distribution platform such that the load exerted through the column member 7 is transferred to the load distribution device and is spread out across the tibia T. The load distribution device may extend from the anterior to the posterior of the tibia T. Alternatively, the load distribution device may extend from the anterior or anterior-medial side of the tibia T into the bone. The load distribution device need not extend all the way through the bone. The load distribution device is fitted by the surgeon making incisions to allow access to, for example, the anterior of the tibia T. Load distributing device receiving bores are then formed, for example, from the anterior of the tibia T to the posterior of the tibia T for receiving the load distribution device.

Although, the invention has been described above in relation to knee joints it may also be used in other joints, for example, elbow joints.

1. A joint device for use as a prosthesis in a human or animal body, the joint device comprising a T-shaped member having a transverse pin fixedly connected to a column member, and a pair of transverse sleeves configured to receive the transverse pin and allow the pin to rotate about its longitudinal axis within the sleeves, wherein the pair of transverse sleeves are configured to engage a bore in a first bone in use, and the column member is configured to be coupled to a second bone in use so that the first and second bones are pivotably interconnected by the joint device.

2. A joint device as claimed in claim 1, wherein the transverse sleeves receive the portions of the transverse pin either side of the region at which the pin is fixedly connected to the column member.

3. A joint device as claimed in claim 1, comprising only one column member for connecting the first and second bones.

4. A joint device as claimed in claim 1, wherein the transverse pin and/or transverse sleeves are configured to limit the rotation of the transverse pin about its own axis within the sleeves.

5. A joint device as claimed in claim 4, wherein the rotation of the transverse pin is limited so that the column member rotates only within a range of 100° in the plane perpendicular to the axis of the transverse pin.

6. A joint device as claimed in claim 1, wherein the transverse sleeves comprise at least one retaining means for fixedly securing them in the bore in said first bone.

7. A joint device as claimed in claim 6, wherein the retaining means comprises a flange portion configured to be screwed into an outer surface of the bone; and/or a rough, knurled or external screw threaded region on the transverse sleeves.

8. A joint device as claimed in claim 1, wherein at least a portion of each transverse sleeve is compressible to allow the transverse pin to rotate/move within the transverse sleeves to a limited extent in directions other than about its own longitudinal axis.

9. A joint device as claimed in claim 8, wherein the transverse sleeves are configured so that the transverse pin can compress the sleeves such that the axis of the transverse pin is able to move/rotate relative to the axis through the sleeves.

10. A joint device as claimed in claim 9, wherein the transverse sleeves are configured so that the transverse pin can compress the sleeves such that the column member is able to rotate to a limited extent about its own longitudinal axis.

11. A joint device as claimed in claim 8, wherein each transverse sleeve comprises an outermost rigid layer or sub-sleeve and an inner compressible layer or sub-sleeve.

12. A joint device as claimed in claim 11, wherein each transverse sleeve further comprises a rigid innermost layer or sub-sleeve configured to receive a portion of the transverse pin.

13. A joint device as claimed in claim 1, further comprising a column sleeve configured to receive a portion of the column member.

14. A joint device as claimed in claim 13, wherein the joint device is configured so that the column member is only able to rotate to a limited extent about its own longitudinal axis within the column sleeve.

15. A joint device for use as a prosthesis in a human or animal body, the joint device comprising a transverse pin, a column member and a pair of transverse sleeves configured to receive the transverse pin, wherein the pair of transverse sleeves are configured to engage a bore in a first bone in use and the column member is configured to be coupled to a second bone in use so that the first and second bones are pivotably interconnected by the joint device, and wherein the transverse sleeves are compressible to allow limited displacement of the rotation axis of the pin in use.

16. A joint device for use as a prosthesis in a human or animal body, the joint device comprising a transverse pin and a column member for pivotably interconnecting two bones in a joint, wherein the joint device further comprises a column sleeve which is configured to engage a bore in one of the bones and to receive a portion of the column member so that the column member can rotate at least to a limited extent about its longitudinal axis within the column sleeve.

17. A joint device as claimed in claim 16, wherein the transverse pin and column member are rigidly interconnected.

18. A joint device as claimed in claim 16, wherein the joint device is configured so that the column member is able to rotate about its own axis within the column sleeve only up to a predetermined angle.

19. A joint device as claimed in claim 18, wherein the column sleeve and/or column member are configured so that the rotation of the column member within the column sleeve is resilient.

20. A joint device as claimed in claim 19, wherein portions of the column member and column sleeve are recessed to provide a cavity between them, and wherein a compressible material is arranged in the cavity so as to provide resilience in the rotation of the column member within the sleeve.

21. A joint device as claimed in claim 16, wherein a portion of the column member comprises a protrusion and a portion of the column sleeve comprises a cavity/slot which receives the protrusion.

22. A joint device as claimed in claim 21, wherein the protrusion and cavity/slot are sized and configured to limit the rotation of the column member about its own axis within the column sleeve.

23. A joint device as claimed in claim 21, wherein the cavity in the sleeve is compressible and co-operates with the protrusion so that the rotation of the column member within the sleeve is resilient.

24. A joint device as claimed in claim 16, wherein the column sleeve is configured so that it has a compressible portion which allows the axis of the column member to pivot/rotate to a limited extent relative to the axis of the column sleeve.

25. A joint device as claimed in claim 16, wherein the column sleeve has an open end for receiving the column member and a closed end upon which the column member rests in use.

26. A joint device as claimed in claim 25, wherein at least a portion of the column sleeve tapers outwardly in a direction towards the sleeve opening.

27. A joint device as claimed in claim 25, wherein the column sleeve comprises a flange portion at least partially surrounding the sleeve opening which is configured such that in use it rests on an outer surface of the bone in which the column sleeve is implanted.

28. A joint device as claimed in claim 16, wherein the joint device comprises a sleeve ring which is configured to co-operate with the column sleeve to distribute the load on the joint device across the bone in which the column sleeve is implanted.

29. A joint device as claimed in claim 16, further comprising an anti-subsidence structure configured to distribute the load on the bone in which the transverse pin is implanted and to prevent subsidence of that bone.

30. A joint device as claimed in claim 29, wherein the anti-subsidence structure comprises a load distribution portion and a tubular body portion which is configured to receive the transverse pin.

31. A method of replacing a human or animal joint comprising forming a first bore in a first bone, inserting a transverse pin into the bore, fixing a pair of transverse sleeves in the bore around portions of the transverse pin, the transverse sleeves and pin being configured to allow the transverse pin to rotate about its own axis, coupling a column member with a second bone to be pivotably interconnected with the first bone, and fixedly interconnecting the transverse pin and column member.

32. A method of replacing a human or animal joint comprising forming a first bore in a first bone, fixing a column sleeve in the bore, inserting a column member into the column sleeve, the column sleeve and column member being configured to allow at least limited rotation of the column member about its own axis, engaging a transverse pin with the column member and a second bone to be pivotably interconnected with the first bone.

33. A method as claimed in claim 32, wherein the first bone is the femur and the first bore is formed above the intercondylar recess.

34. A method as claimed in claim 33, wherein a second bore is formed from the intercondylar recess at least part way into the femur so that it intersects with the first bore.

35. A method as claimed in claim 34, wherein the transverse pin and column member are engaged with each other so that column member is pivotable in the region at which the first and second bores interconnect.

36. A method as claimed in claim 33, wherein the transverse pin is mounted in the first bore formed in the femur such that a portion of the length of the transverse pin forms the upper surface of the intercondylar recess.
